(19) United States
(12) Patent Application Publication
(10) Pub. No.: US 2010/0036286 A1
(43) Pub. Date: Feb. 11, 2010

(54) DEVICE FOR INDIRECTLY MEASURING OCCLUSAL FORCES

(76) Inventors: Alexander Scholz, Kirchweihdach (DE); Johannes Clauss, Munchen (DE)

Correspondence Address: D. PETER HOCHBERG CO. L.P.A., 1940 EAST 6TH STREET, CLEVELAND, OH, 44114, US

(21) Appl. No.: 12/449,789

(22) PCT Filed: Feb. 22, 2008
(86) PCT No.: PCT/EP2008/001442

§ 371 Date: Oct. 21, 2009

(30) Foreign Application Priority Data
Feb. 28, 2007 (DE) 10 2007 009 910.1

(51) Int. Cl. A61B 5/11

(52) U.S. Cl. 600/590

(57) ABSTRACT

An apparatus, in particular a dental splint, for the measurement of occlusal forces in case of a terminal occlusion of teeth comprises at least one sensor which is arranged outside of an occlusion region of the apparatus. A system for the diagnosis or treatment of bruxism serves to determine the position or the size of occlusal forces on the apparatus. A method allows exact localization and quantitative determination of occlusal forces on the apparatus.

This application is a National Stage application of International Application No. PCT/EP2008/001442, filed on Feb. 22, 2008 and claims priority of German Application Serial No. DE 10 2007 009 910.1 filed Feb. 27, 2007.

### BACKGROUND OF THE INVENTION

1. Field of the Invention

The invention relates to an apparatus, in particular a dental splint, comprising at least one sensor, for the measurement of occlusal forces in case of a terminal occlusion of teeth. Furthermore, the invention relates to a system for the diagnosis or treatment of bruxism. In addition, the invention relates to a method for the determination of the position or the size of occlusal forces on the apparatus.

2. Description of the Prior Art

Dental splints which can be attached to one or a plurality of teeth and are intended to assume various tasks have already been used within the scope of various diagnostic or therapeutic methods in medicine and/or dentistry. Diverse dental splints are, in particular, used in the treatment of bruxism (colloquially also referred to as “grinding of teeth” or “clenching of teeth”).

A bruxism patient carries out a parafunctional clenching of the teeth which usually takes place unknowingly. Often, a strong force is exerted to bring about an occlusion of the teeth, i.e. a terminal occlusion of the upper and lower teeth, during the bruxism incidents occurring during the day and the night. In addition to disturbing effects, such as grinding of the teeth, this also results in severe direct and indirect pathological consequences for the patient, for example wear of the teeth, overload of the periodontium, the mandibular joint and the masticatory muscles, tinnitus or vertigo.

Apart from purely mechanical dental splints for protecting the teeth from wear, electronic dental splints in which sensors for measuring the occlusion pressure are arranged have also been developed for the treatment of bruxism. Herein, measurement values can be output to an appropriate receiver unit via cable, said measurement values being used to carry out a diagnostic data evaluation. What is more, wireless transmission of data to a receiver unit has already been realized via a telemetric path, said data being taken up by electronics arranged in the dental splint.

Forces on the teeth, which occur during bruxism incidents or other parafunctions of the teeth and/or the muscles of the jaws can, therefore, be collected and analyzed. For example, U.S. Pat. No. 5,078,153 discloses a dental splint wherein a piezoelectric film acting as a sensor is positioned between the occlusal surfaces of the molar teeth, so as to take up an occlusion pressure on the occlusal surfaces of the teeth. U.S. Pat. No. 5,586,562 discloses an apparatus for taking up an occlusion pressure wherein an electric sensor is arranged above the occlusal surface of a tooth to collect information about an occlusion pressure there.

The existing electronic dental splints for the diagnosis or treatment of bruxism have in common that an occlusion pressure is always measured by an electric sensor which is arranged within the occlusion zone of the teeth, that is in that zone in which opposite teeth of the upper jaw and the lower jaw contact each other in a terminal occlusion position.

In the following, an occlusion region is to be interpreted as that area of the dental splint which, if the splint is properly used in case of an occlusion of teeth 11 to 18 and/or 21 to 28 (according to the Federation Dentaire International, FDI) of the upper jaw or of teeth 31 to 38 and/or 41 to 48 of the lower jaw (also referred to as “upper” and “lower” teeth below), come into contact with a tooth, that means essentially those regions of the splint that antagonize the occlusal surfaces of the teeth.

U.S. Pat. No. 5,846,211 A discloses a measuring device for occlusal forces wherein an occlusion force is taken up across a pressure transmitting region arranged in an occlusion region of teeth and is transferred to a pressure detector. A grip for holding the apparatus is formed such that it is arranged outside of a test person's oral cavity and can be held there in case of an occlusion by said test person. The occlusion is effected directly on a pressure takeup disk and the pressure transmitting region.

XP 000175116, Blamphin C. N. J; Branfield T. R.; Jobbins B.; Fisher J.; Watson C. J.; Redfen E. J., “A simple instrument for the measurement of maximum occlusal force in human dentition”, Proceedings of the Institution of Mechanical Engineers, Part H (Journal of Engineering in Medicine), 1990, UK, ISSN 0954-4119, vol. 204, no. H2, pg. 129-131, discloses a dental apparatus for the measurement of a maximum occlusion force. A terminal occlusion is effected on polymer regions of a two-member frame which is held together by a hinge. A force transducer is arranged between the two members of the frame, so that said force transducer can take up a force transferred via the frame.

The invention aims at presenting an apparatus for the measurement of occlusal forces as well as a method for the evaluation of measured variables.

### SUMMARY OF THE INVENTION

In an apparatus according to the invention for the measurement of occlusal forces in case of a terminal occlusion of teeth, at least one sensor is provided which is arranged outside of the occlusion zone of the teeth. Herein, occlusal forces are to be interpreted as all of those forces which may develop between teeth of the upper jaw and the lower jaw in case of an occlusion which is also referred to as “terminal occlusion” or “intercuspidation”. In case of a terminal occlusion, these forces may have different directions according to the moving direction of the teeth. For example, an occlusion may be a static occlusion, a habitual occlusion, a centric occlusion, or a maximum intercuspidation and is, furthermore, not limited to one of these or further potential types of occlusion but can also be a mixed type consisting of different occlusion types.

An occlusion zone of the teeth is to be interpreted as the region between upper and lower teeth in case of a terminal occlusion, which is positioned between the occlusion points of the upper and lower teeth, that is between those points where the upper and lower teeth contact each other in case of a terminal occlusion. Therefore, the occlusion zone of the teeth is, in essence, the zone which is positioned between the occlusal surfaces of the upper and lower teeth. However, the occlusion zone also comprises such zones between the upper and lower teeth where occlusion points of teeth of the upper jaw and the lower jaw are positioned opposite to each other, said occlusion points being induced by any type of occlusion. Accordingly, the occlusion zone can also be interpreted as the projection zone of the occlusal surfaces of the upper and lower teeth in relation to each other.

Therefore, the provided sensor is not arranged between the occlusal surfaces of the upper and lower teeth but outside of the zone between the occlusal surfaces, that is outside of the occlusion zone of the teeth. For example, a sensor can be arranged to the side of the molar teeth or in front of or behind the incisor teeth. In other words, the sensor is arranged outside of a region of intersection between the occlusion plane (defined as the plane through the connecting lines between the point of contact of the cutting edges of the lower central incisor teeth 31 and 41 and what is called the “distal cusp” of teeth 36 and 46 through the lip closure line) and the occlusion zone of the teeth. If the arrangement comprises a plurality of sensors, each of these sensors is, accordingly, arranged outside of the occlusal surfaces and the zone between the occlusal surfaces.

In an embodiment of the present invention and according to the invention, a sensor can be arrangeable on the outside of a tooth, for example on the dental enamel of a tooth or on the dental cement thereof, or even on the outside of a dental filling, a plombage, a tooth crown, or a dental inlay. The sensor can, likewise, be arrangeable on the outside of a dental implant, an artificial tooth, or a dental prosthesis. As regards the above-mentioned cases, it is of no relevance whether the sensor is arrangeable on the outer side of the teeth which is facing away from the tongue or on the inner side of the teeth which is facing the tongue, as long as the sensor is not arranged in the occlusion zone of the teeth according to the description above.

In a further embodiment, the sensor can also be arrangeable inside a tooth, for example inside the dental enamel thereof or inside the dental cement thereof, or even inside a dental filling, a plombage, a tooth crown, or a dental inlay. The sensor can, likewise, be arrangeable inside a dental implant, an artificial tooth, or a dental prosthesis.

The apparatus according to the invention provides a support which comprises an occlusion region. Herein, an occlusion region is to be interpreted as a coherent or incoherent region of the support which, on the one hand, comprises the zones of occlusion of the upper teeth with the support and of the lower teeth with the support. On the other hand, the occlusion region of the support comprises at least one region between the zones of occlusion of the upper or lower teeth, respectively, with the support. According to the descriptions above, an occlusion region of the support is positioned between the occlusal surfaces of the upper and lower teeth or within an occlusion area of the teeth. Such a definition of the occlusion zones and occlusion regions is necessary because it allows differentiating between an occlusion of teeth, i.e. the occlusion in its actual medical sense, and an occlusion of teeth with a support positioned therebetween.

In the latter case, there is no direct antagonistic contact between upper and lower teeth because the support prevents a touch contact. But there is an occlusion, i.e. a terminal occlusion contact, between upper teeth and the support as well as an occlusion between lower teeth and the support. These upper and lower occlusion zones of the support and the zones of the support positioned between these occlusion zones define the occlusion region of the support.

According to the arrangement corresponding to the embodiments described above, a sensor of the apparatus is arranged outside of the occlusion region of the support, i.e. outside of an area of the support where the occlusal surfaces of the upper and lower teeth are arranged opposite to each other in case of a terminal occlusion.

In an embodiment of the invention, the occlusion region of the support is, in essence, arrangeable in parallel to an occlusal surface of the teeth. At least one occlusal surface of the upper or the lower teeth can, therefore, be covered by the support. As a matter of course, both an occlusal surface of the upper teeth and an occlusal surface of the lower teeth should be covered by the support in case of a terminal occlusion between upper jaw and lower jaw, with the result that an occlusion of the upper and lower teeth, respectively, with the support is achieved even if the support is only fitted onto teeth of the upper or the lower jaw. In this embodiment of the invention, the support furthermore possesses a flank region which is, in essence, arrangeable perpendicular to an occlusal surface of the upper or lower teeth. A flank region is, thus, a region of the support which is positioned to the sides of the molar, canine or incisor teeth and outside of an occlusion zone of the teeth.

The support may be a plastic splint such as it is also referred to as “dental splint”, which can be fitted onto one tooth or a plurality of teeth. Therein, the splint can be clamped, clipped, pushed or attached onto at least one tooth of the upper jaw or the lower jaw in any other detachable or non-detachable manner. It is also possible that the plastic splint is attachable to at least one tooth of the upper jaw as well as to at least one tooth of the lower jaw. The material of the plastic splint may, in particular, be a dental plastic material which is non-poisonous and abrasion-resistant. Advantageous use can also be made of a plastic material which is mainly transparent once it has hardened. But it is also possible to use any other plastic or further materials.

In an embodiment of the present invention, a sensor is completely arranged inside the support such that the sensor is completely enclosed by the support and does not project therefrom or is positioned on an outside of the support.

It is also possible to arrange a sensor or a plurality of sensors within the flank region of the support, for example in the region of the support positioned behind an incisor tooth or to the side of a molar tooth.

A sensor registers a physical force by sensorizing scalar quantities generated by the effect of the force. A sensor can, thus, be an expansion sensor for sensorizing an expansion, a bending sensor for sensorizing a bend, a vibration sensor for sensorizing a vibration, a tension sensor for sensorizing a mechanical tension or restraint, a displacement sensor for sensorizing a mechanical displacement, or a pressure sensor for sensorizing a pressure. As a matter of course, however, it is also possible to use a sensor for directly sensorizing directed physical quantities, such as force sensors for sensorizing the amount and direction of a force.

In an embodiment of the apparatus according to the invention, a sensor is a piezoelectric sensor for converting a mechanical pressure into an electrical voltage. According to the invention, a sensor can also be a strain gauge for converting a mechanical bend into the change of an ohmic resistance.

In a further embodiment of the invention, a plurality of sensors is provided which can possess the same function or different functions, for example sensorizing different physical quantities or having different structures. For example, it is also possible to use sensors having different structures for sensorizing the same quantities according to the same measuring principle or according to different measuring principles.

In an embodiment of the invention, an electronic unit is provided which can take up or evaluate measured data registered by a sensor. To achieve this, the electronic unit is, preferably, conductively connected to a sensor. It is, however, also possible to provide a wireless connection between a sensor and the electronic unit. The electronic unit can be arranged in a flank region of the support to the side of molar teeth. Furthermore, the electronic unit can also comprise a storage unit for storing measured variables.

In a further embodiment of the present invention, the apparatus comprises a transmitter unit for the transmission of measured variables which were or are registered by a sensor or were or are taken up or evaluated by the electronic unit.

The support can include at least two different regions with different physical and/or mechanical properties, such as hardness, stiffness or strength. For example, the support can consist of two or more different types of plastic material having a different hardness.

A least two sensors can be completely arranged within one of each of the different regions having different mechanical properties, with the result that, for example, one sensor is arranged within a first region of the support having a first hardness and a second sensor is arranged within a region of the support having a second hardness. This arrangement allows drawing comparisons between the effects of forces on the different regions, wherein said comparisons can be used for the evaluation of the measured data and, in particular, for the localization and the quantitative determination of a pressure. To achieve this according to the invention, at least two sensors can also be arranged adjacent to at least two different regions having different physical and/or mechanical properties, so that they are not completely positioned within the different regions.

It is possible to determine the position and the size of occlusal forces although a sensor is not arranged in an occlusion zone of the teeth or an occlusion region of the support. With the appropriate method according to the invention, at least two sensors are provided inside a support, however outside of an occlusion region of the support. Preferably, the sensors are distributed along the occlusion region of the support such that forces can be registered across the entire occlusion region and the occlusion region does, therefore, not comprise any regions where forces cannot be registered by one of the sensors provided. If a bruxism incident occurs, for example a pressure on a point in the occlusion region, the sensors register the pressures occurring independently of each other. If three sensors are provided, three intensity values of the pressure are obtained. A quantitative determination of the occurring occlusion pressure is achieved by a comparison of the amplitudes, i.e. the intensity values of the individual sensors. A localization of the occurring bruxism incident is also achieved by a comparison of the amplitudes, i.e. the intensity values of the individual sensors. The higher the amplitudes of the measured values at a sensor, the stronger the basic pressure or the closer a force effect to the corresponding sensor. Vice versa, a sensor registers the consequences of a weak force effect or a distant effect as a weaker measurement signal. If the material properties of the support are known, it is, therefore, possible to determine an exact size of an occurring force. If, for example, three sensors are provided which are arranged on a same side of the occlusion region, each distribution of the measured intensities corresponds to a point along a one-dimensional curve within the occlusion region of the support. The corresponding position of a bruxism incident along this curve can, therefore, be concluded from an amplitude spectrum of the three sensors.

A more precise localization of an occurring pressure by providing a plurality of sensors inside the support and outside of the occlusion region of the support can be achieved by arranging the sensors around the occlusion region or on different ones of its sides. Then, each distribution of the amplitudes, i.e. of the intensities of the registered forces, corresponds to a point not along a one-dimensional curve but in a two-dimensional plane in the occlusion region. If the intensities of all sensors are examined at the same time, the intensity distribution or the intensity spectrum of the sensors can be interpreted as a signature for a specific point.

Apart from the comparison of the intensities or amplitudes of the forces measured in the occlusion region of the support by the sensors, it is, according to the above descriptions, also possible to compare the runtime differences between the sensors for localizing occlusal forces. The runtime of a distant bruxism incident to a sensor is longer than the runtime of a close bruxism incident. The provision of a plurality of sensors, therefore, allows a comparison between the runtime differences for the registration of the measured variables by the sensors, in order to localize an occlusal force and to determine its size.

With these described methods for the localization and quantitative determination of occlusal forces, it is also possible to determine a plurality of simultaneously occurring occlusal forces at a plurality of occlusion points in the occlusion region of the support. According to the invention, this can be achieved by comparing both the amplitudes and the runtime differences, in order to thus provide additional parameters for the determination of the position and size of occlusal forces.

### BRIEF DESCRIPTION OF THE DRAWINGS

Below, these and further features and advantages of the invention are illustrated in more detail in exemplary embodiments, with reference being made to the drawings. In the drawings,

FIGS. 1A and 1B show schematic diagrams of an apparatus according to the invention in a first embodiment of the invention;

FIGS. 1C to 1F show schematic diagrams of occlusion points, occlusion zones and occlusal surfaces of teeth;

FIG. 2A shows a schematic diagram of an apparatus according to the invention in a further embodiment of the invention;

FIG. 2B shows a schematic diagram of an apparatus according to the invention in a further embodiment of the invention;

FIG. 2C shows a schematic diagram of an apparatus according to the invention in a further embodiment of the invention;

FIG. 2D shows a schematic diagram of an apparatus according to the invention in a further embodiment of the invention;

FIG. 3 shows a schematic diagram of an apparatus according to the invention in a further embodiment of the invention;

FIG. 4 shows a schematic diagram of an apparatus according to the invention in a further embodiment of the invention;

FIG. 5A shows a schematic diagram of an apparatus according to the invention in a further embodiment of the invention;

FIG. 5B shows an intensity diagram of sensors of the embodiment of the invention of FIG. 5A;

FIG. 5C shows a schematic diagram of an apparatus according to the invention in a further embodiment of the invention.

### DETAILED DESCRIPTION OF THE PREFERRED EMBODIMENTS

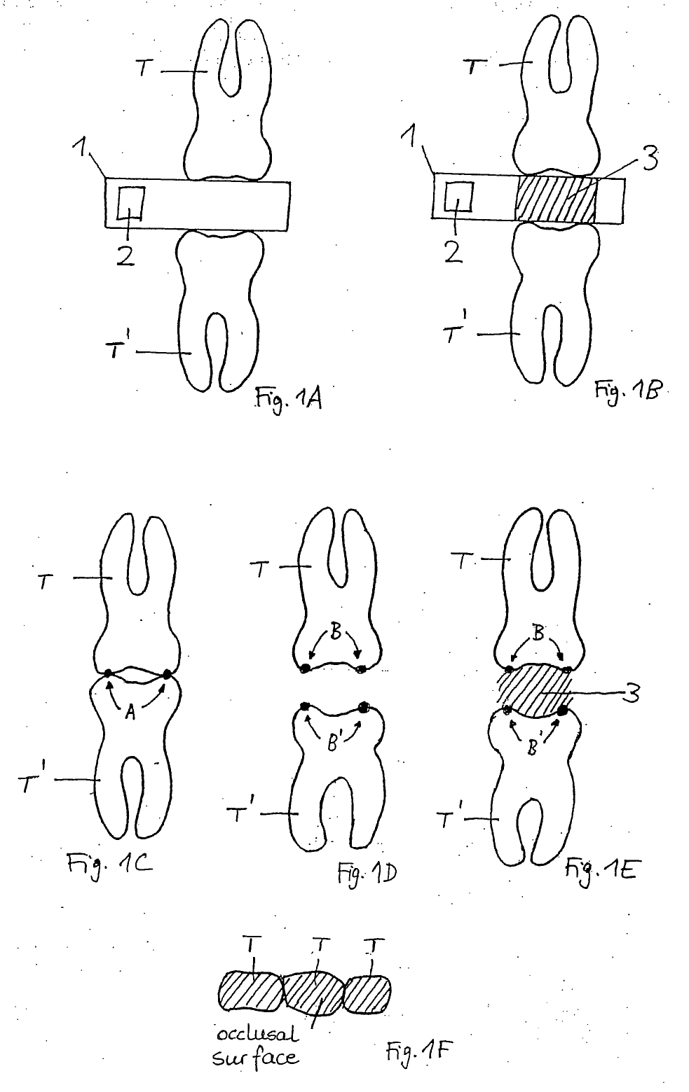

FIG. 1A shows a schematic diagram of an apparatus 1 according to the invention for the measurement of occlusal forces in case of a terminal occlusion of teeth comprising a sensor 2. The figure shows the position of a tooth T, T′ each of the upper jaw and the lower jaw in case of a terminal occlusion with the apparatus 1.

FIG. 1B shows the apparatus 1 according to FIG. 1A and, furthermore, illustrates that the sensor 2 is arranged outside of an occlusion zone 3 of the teeth T, T′. To provide a better understanding of the occlusion zone 3, reference is made to the following figures, in particular to FIG. 1E.

To illustrate the term occlusion zone, FIG. 1C shows two antagonistic teeth T, T′ in case of a terminal occlusion. Herein, points A represent the occlusion points of the teeth T, T′, i.e. those points where the upper and the lower teeth T, T′ come into contact in case of a terminal occlusion. We speak of occlusion points although the zone of antagonistic contact may, of course, also have the shape of an area. In the following, we nevertheless speak of occlusion points in accordance with current nomenclature, which are, however, also to be interpreted as extended contact areas of the teeth.

FIG. 1D shows two antagonistic teeth with the exemplary occlusion points B of the upper tooth T and the occlusion points B′ of the lower tooth T′. Although there is no occlusion in the tooth position shown, the points B, B′ are referred to as occlusion points.

FIG. 1E shows the two teeth of FIG. 1D with the occlusion points B, B′ of the teeth T, T′. Furthermore, the figure shows an occlusion zone 3 which is the zone between the upper and lower teeth T that is enclosed by the occlusion points B, B′ and is, thus, positioned between the occlusion points B, B′. According to general linguistic usage, the occlusion zone 3 can also be described as the zone which is positioned between the occlusal surfaces of the upper and lower teeth T, T′.

For illustration purposes, FIG. 1F shows a top view of the occlusal surface of three adjacent teeth T of one row of teeth.

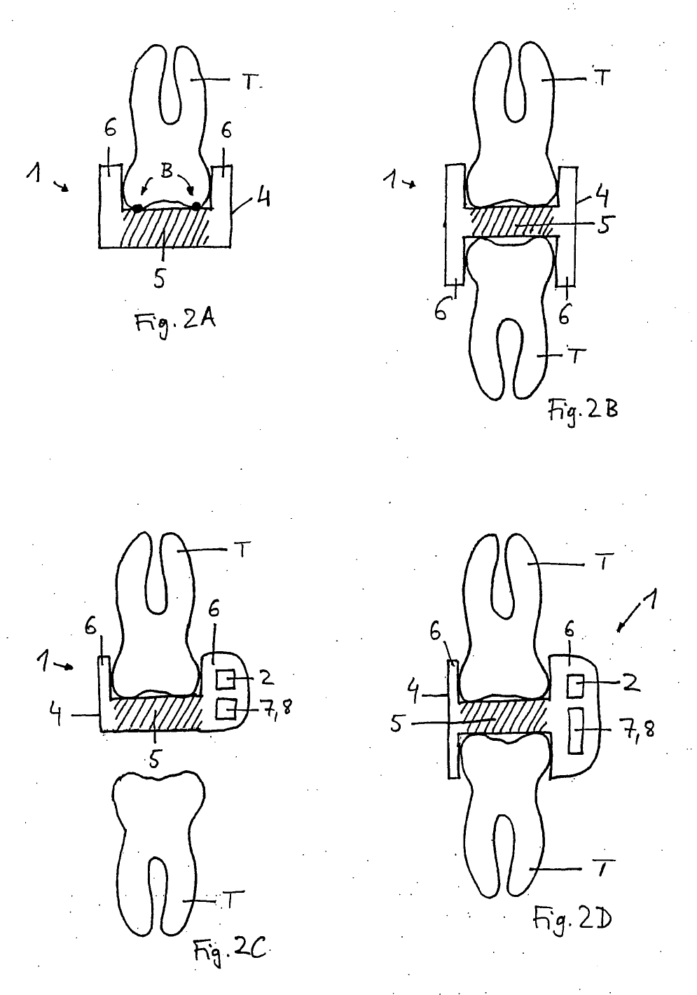

FIG. 2A shows a schematic diagram of an embodiment of the present invention wherein the apparatus 1 comprises a support 4. The support 4 comprises an occlusion region 5 (hatched) which, as shown, comprises both the region of the carrier 4 that abuts against occlusion points B and the occlusal surface of a tooth T as well as the part of the support 4 in a vertical projection region of the occlusal surface. In other words, the occlusion region 5 of the support 4 is defined as the region of the support 4 that is arranged above or below the occlusal surface of a tooth T. The occlusion region 5 of the support 4 is, therefore, the part of the support 4 which is, in essence, arranged in parallel to the occlusal surface of a tooth T. In addition to an occlusion region 5, the support 4 comprises flank regions 6 which are, in essence, arranged perpendicular to the occlusal surface of the tooth T. A flank region 6 is, therefore, positioned outside of an occlusion region 5 and, thus, also outside of an occlusion zone of the teeth T. By providing an occlusion region 5 and flank regions 6 of the support 4, the apparatus 1 can be fitted onto a tooth T as shown.

FIG. 2B shows a schematic diagram of a further embodiment, similar to FIG. 2A, with a support 4 which comprises an occlusion region 5 as well as flank regions 6. In this embodiment, the flank regions 6 are formed such that they are arranged to the side not only of one tooth T but to the side of an upper tooth T and a lower tooth T′. As shown, the apparatus 1 can, therefore, be simultaneously fitted onto a tooth T of the upper row of teeth and onto a tooth T′ of the lower row of teeth.

FIG. 2C shows a schematic diagram of the arrangement of a sensor 2 in an apparatus 1 according to the present invention. The sensor 2 is arranged in a flank region 6 of the support 4 and outside of an occlusion region 5 of the support. Furthermore, FIG. 2C shows the arrangement of an electronic unit 7 as well as a transmitter unit 8 in a flank region 6 of the support 4. The flank regions 6 of the support 4 are formed such that the arrangement can be fitted onto at least one tooth T of the upper teeth or onto at least one tooth T′ of the lower row of teeth. In the illustrated instance, the apparatus 1 is attached to at least one tooth T of the upper jaw.

FIG. 2D shows a schematic diagram of the arrangement of a sensor 2 in an apparatus 1 according to the present invention. The sensor 2 is arranged in a flank region 6 of the support 4 and outside of an occlusion region 5 of the support. Furthermore, FIG. 2D shows the arrangement of an electronic unit 7 as well as a transmitter unit 8 in a flank region 6 of the support 4. In this embodiment, the flank regions 6 of the support 4 are formed such that the arrangement can be fitted onto at least one tooth T of the upper teeth or onto at least one tooth T′ of the lower row of teeth.

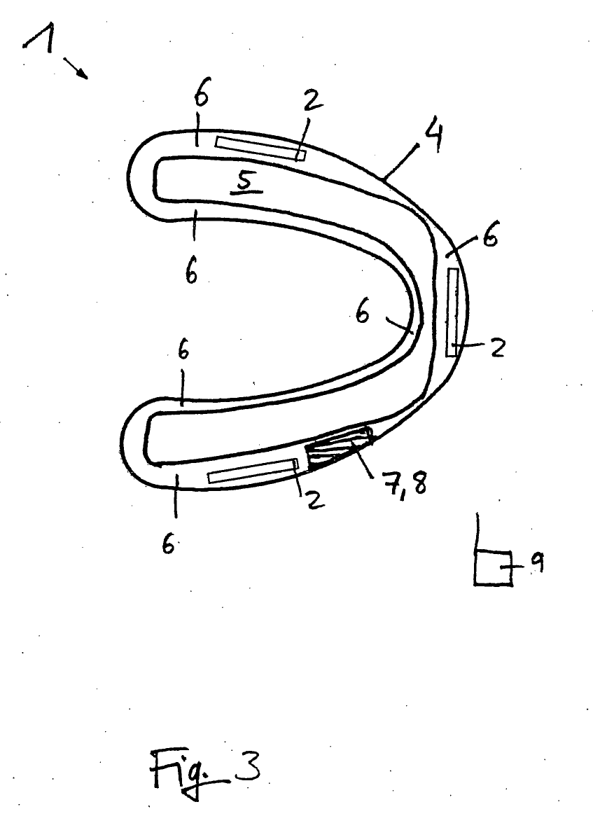

A schematic diagram of a corresponding embodiment of the apparatus 1 is shown in FIG. 3. Three sensors 2 are arranged inside the support 4, however outside of an occlusion region 5 of the support 4. As shown, the three sensors are arranged in a flank region 6 of the support 4. An electronic unit 7 which can take up or evaluate the measured data registered by a sensor is additionally arranged in one of the flank regions 6 of the support 4. A transmitter unit 8 which can transmit the measured variables which were or are registered by a sensor 2 or were or are taken up or evaluated by the electronic unit 7 to a receiver unit 9 is, likewise, arranged in a flank region 6 of the support 4. As shown, the receiver unit 9 is arranged outside of the apparatus 1. It serves to receive the measured data and can transfer said data to a further unit or prepare, process or evaluate said data by means of an implemented signal processing unit.

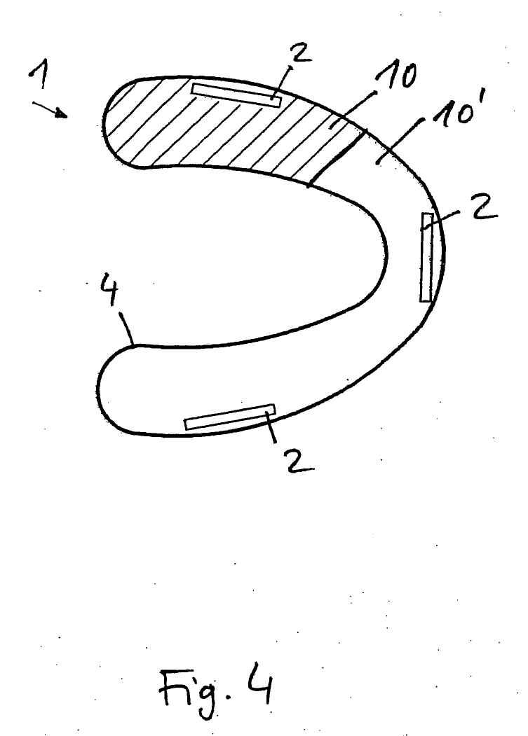

FIG. 4 shows a schematic diagram of a further embodiment of the invention wherein three sensors 2 are arranged in the support 4 of the apparatus 1. The support 4 consists of two regions 10, 10′ having different mechanical properties, such as hardness or stiffness. A sensor 2 is arranged inside one region 10 while two sensors 2 are arranged inside the other region 10′. As long as the corresponding mechanical coefficients and/or properties of the different regions 10, 10′ are known, data processing of the values measured by the sensors can, in this manner, be expanded by parameters.

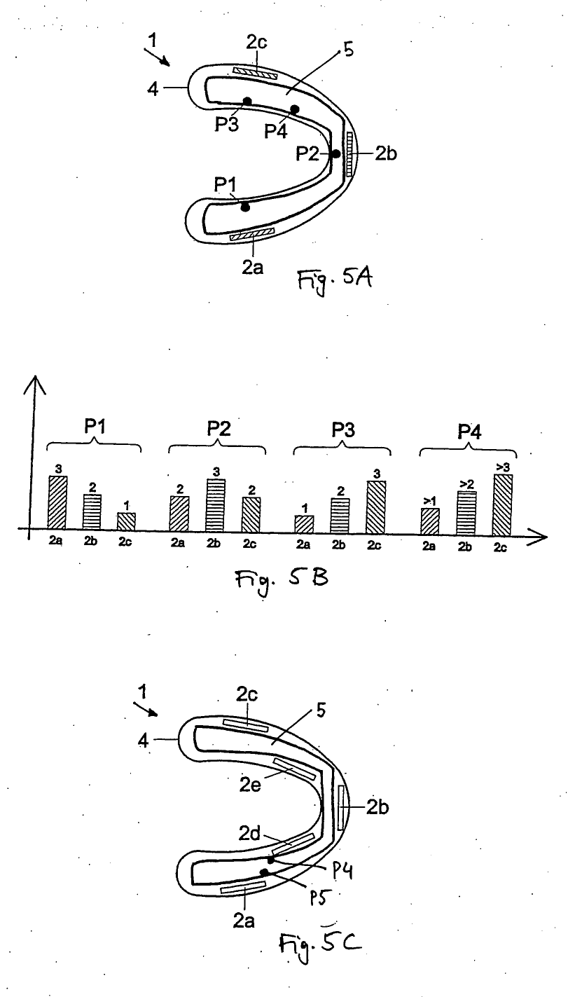

FIG. 5A shows a schematic diagram of an apparatus 1 for the measurement of occlusal forces in case of a terminal occlusion of teeth comprising three sensors 2a, 2b, 2c, which are arranged inside the support 4 of the apparatus 1 and outside of the occlusion region 5. By arranging the sensors 2a, 2b, 2c along the occlusion region 5, a bruxism incident, for example a pressure on a point P1, P2, P3 or P4 in the occlusion region 5, can be localized along a one-dimensional curve. To achieve this, the pressure intensities measured at the sensors 2a, 2b, 2c can be compared, as is shown in FIG. 5B in an exemplary manner. Each distribution of the intensities 1, 2, 3 (ordinate) measured at the sensors 2a, 2b, 2c corresponds to a specific point along a line in the occlusion region 5. If seen in combination, the first three values 3, 2, 1 for the intensities of the sensors 2a, 2b, 2c reflect an intensity signature for a point P1 according to FIG. 5A. If seen in combination, the second, third and forth value triples 2, 3, 2 and 1, 2, 3 and >1, >2, >3 of the intensities of the sensors 2a, 2b, 2c, accordingly, reflect the intensity signatures for the points P2, P3 and P4 of FIG. 5A.

FIG. 5C shows a schematic diagram of an apparatus 1 for the measurement of occlusal forces according to a further embodiment of the invention. Five sensors 2a to 2e are arranged inside the support 4 of the apparatus 1 and outside of the occlusion region 5 of the support 4. By arranging the sensors 2a to 2e around the occlusion region 5 of the support 4 or the apparatus 1, a bruxism incident, for example a pressure on a point P4, P5 in the occlusion region 5, can be localized within a two-dimensional area. To achieve this, the pressure intensities measured at the sensors 2a to 2e or runtime differences of the sensor measurements are compared.

Having described preferred embodiments of the invention, it will be apparent to those skilled in the art to which this invention relates, that modifications and amendments to various features and items can be effected and yet still come within the general concept of the invention. It is to be understood that all such modifications and amendments are intended to be included within the scope of the present invention.

1. A dental splint for the measurement of occlusal forces in case of a terminal occlusion of teeth, wherein said dental splint is provided for coming into contact with an occlusal surface of the teeth, said dental splint comprising:
at least one sensor for determining the measurement of the occlusal forces;
a support comprising:
two opposing flank regions arrangeable to be perpendicular to the occlusal surface of the teeth; and
an occlusion region arrangeable to be parallel to the occlusal surface of the teeth;
wherein the support can be fitted onto at least one of the teeth by means of the flank regions and the occlusion region;
wherein the at least one sensor is arranged within one of the flank regions of the support and outside of the occlusion region, the support completely enclosing the at least one sensor.

2. The dental splint according to claim 1, wherein the at least one sensor is arrangeable on the outside of a tooth, on the outside of a dental filling, a plombage, a crown or an inlay of a tooth, or on the outside of a dental implant, an artificial tooth or a dental prosthesis.

3. The dental splint according to claim 1, wherein the at least one sensor is selected from the group consisting of an expansion sensor, a bending sensor, a vibration sensor, a tension sensor, a displacement sensor, a pressure sensor, and a force sensor.

4. The dental splint according to claim 1, wherein the at least one sensor is a piezoelectric sensor or a strain gauge.

5. The dental splint according to claim 1, wherein said at least one sensor is a plurality of sensors differing in structure or differing in function.

6. The dental splint according to claim 1, and further comprising an electronic unit for taking up and/or evaluating measured variables registered by the at least one sensor.

7. The dental splint according to claim 6, and further comprising a transmitter unit operatively connected to the at least one sensor for transmitting measured variables registered by the at least one sensor or taken up or evaluated by the electronic unit to a receiver unit.

8. The dental splint according to claim 1, wherein the support comprises at least two different regions, each region having a different hardness, stiffness or strength.

9. The dental splint according to claim 8, wherein the at least one sensor is a plurality of sensors completely arranged within either one, the other or both of the at least two different regions.

10. The dental splint according to claim 8, wherein the at least one sensor is a plurality of sensors arranged adjacent to either one, the other or both of the at least two different regions.

11. A system for the diagnosis or treatment of bruxism, comprising:
a dental splint in case of a terminal occlusion of teeth, wherein said dental splint is provided for coming into contact with an occlusal surface of the teeth, said dental splint comprising:
at least one sensor for determining the measurement of the occlusal forces; and
a support comprising:
two opposing flank regions arrangeable to be perpendicular to the occlusal surface of the teeth; and
an occlusion region arrangeable to be parallel to the occlusal surface of the teeth;
wherein the at least one sensor is arranged within one of the flank regions of the support and outside of the occlusion region, the support completely enclosing the at least one sensor;
a transmitter unit for transmitting measured variables registered by the at least one sensor; and
a receiver unit for receiving measured variables transmitted by the transmitter unit.

12. A method for the determination of the position or size of occlusal forces on a dental splint in case of a terminal occlusion of teeth, wherein said dental splint is provided for coming into contact with an occlusal surface of the teeth, said dental splint comprising:
at least two sensors for determining the measurement of the occlusal forces; and
a support comprising:
two opposing flank regions arrangeable to be perpendicular to the occlusal surface of the teeth; and
an occlusion region arrangeable to be parallel to the occlusal surface of the teeth;
an electronic unit for taking up and/or evaluating measured variables registered by the at least two sensors;
said method comprising the steps of:
providing said at least two sensors by arrangement inside the support or inside or on the outside of a tooth, on the outside of a dental filling, a plombage, a crown or an inlay of a tooth, or on the outside of a dental implant, an artificial tooth or a dental prosthesis; and
localizing or quantitatively determining the occlusal forces by the electronic unit by comparing amplitudes or runtime differences of measured variables which the at least two sensors provide to the electronic unit.
